SEQUENCE LISTING (29 sequences)

SEQ ID NO: 1 - protein, 10 aa, Artificial
  Random inserted peptide sequence in landscape phage library
EHWSYGLRPG

SEQ ID NO: 2 - protein, 4 aa, Artificial
  Random inserted peptide sequence in landscape phage library
EHWS

SEQ ID NO: 3 - protein, 9 aa, Artificial
  Random inserted peptide sequence in landscape phage library
EHPAGMTGD

SEQ ID NO: 4 - protein, 9 aa, Artificial
  Random inserted peptide sequence in landscape phage library
EWSSSSTDT

SEQ ID NO: 5 - protein, 9 aa, Artificial
  Random inserted peptide sequence in landscape phage library
ATDTRPGTE

SEQ ID NO: 6 - protein, 10 aa, Artificial
  Random inserted peptide sequence in landscape phage library
EHWSYGLDPA

SEQ ID NO: 7 - protein, 10 aa, Artificial
  Random inserted peptide sequence in landscape phage library
EHWSYGLDPA

SEQ ID NO: 8 - protein, 10 aa, Artificial
  Random inserted peptide sequence in landscape phage library
EHWSYGLSPA

SEQ ID NO: 9 - protein, 10 aa, Artificial
  Random inserted peptide sequence in landscape phage library
EHWSYGLRPG

SEQ ID NO: 10 - protein, 8 aa, Artificial
  Random inserted peptide sequence in landscape phage library
EHWSGGGS

SEQ ID NO: 11 - protein, 9 aa, Artificial
  Random inserted peptide sequence in landscape phage library
EHWSYGGGS

SEQ ID NO: 12 - protein, 7 aa, Artificial
  Random inserted peptide sequence in landscape phage library
EHWGGGS

SEQ ID NO: 13 - protein, 8 aa, Artificial
  Random inserted peptide sequence in landscape phage library
GLRPGGGS

SEQ ID NO: 14 - protein, 7 aa, Artificial
  Random inserted peptide sequence in landscape phage library
GRPGGGS

SEQ ID NO: 15 - protein, 6 aa, Artificial
  Random inserted peptide sequence in landscape phage library
GRPGGS

SEQ ID NO: 16 - protein, 5 aa, Artificial
  Random inserted peptide sequence in landscape phage library
GRPGS

SEQ ID NO: 17 - protein, 8 aa, Artificial
  Random inserted peptide sequence in landscape phage library
EHWSLRPG

SEQ ID NO: 18 - protein, 10 aa, Artificial
  Random inserted peptide sequence in landscape phage library
GHWSYGLGGS

SEQ ID NO: 19 - protein, 8 aa, Artificial
  Random inserted peptide sequence in landscape phage library
EHWSKDMV

SEQ ID NO: 20 - protein, 8 aa, Artificial
  Random inserted peptide sequence in landscape phage library
EHWSRPAL

SEQ ID NO: 21 - protein, 8 aa, Artificial
  Random inserted peptide sequence in landscape phage library
EFRPGEHV

SEQ ID NO: 22 - protein, 8 aa, Artificial
  Random inserted peptide sequence in landscape phage library
EWSYKLRA

SEQ ID NO: 23 - protein, 9 aa, Artificial
  Random inserted peptide sequence in landscape phage library
ELRPGEKVL

SEQ ID NO: 24 - protein, 8 aa, Artificial
  Random inserted peptide sequence in landscape phage library
EHWKEAVL

SEQ ID NO: 25 - protein, 8 aa, Artificial
  Random inserted peptide sequence in landscape phage library
ELRPGEHV

SEQ ID NO: 26 - protein, 8 aa, Artificial
  Random inserted peptide sequence in landscape phage library
EHWSRELV

SEQ ID NO: 27 - protein, 8 aa, Artificial
  Random inserted peptide sequence in landscape phage library
ELRPGEHV

SEQ ID NO: 28 - protein, 8 aa, Artificial
  Random inserted peptide sequence in landscape phage library
EHWKEAVL

SEQ ID NO: 29 - protein, 10 aa, Artificial
  Random inserted peptide sequence in landscape phage library
EHWSYGLRPG

(19) United States
(12) Patent Application Publication
(10) Pub. No.: US 2012/0156215 A1
(43) Pub. Date: Jun. 21, 2012

(54) Phage-GnRH Constructs, Their Mimics, Antigenic Compositions, and Sequences for Immunocontraception of Animals and Other Applications

(75) Inventors: Tatiana I. Samoylova, Auburn, AL (US); Valery A. Petrenko, Auburn, AL (US); Nancy R. Cox, Auburn, AL (US); Alexandre M. Samoylov, Auburn, AL (US)

(73) Assignee: Auburn University, Auburn, AL (US)

(21) Appl. No.: 13/165,422

(22) Filed: Jun. 21, 2011

Related U.S. Application Data
(60) Provisional application No. 61/356,858, filed on Jun. 21, 2010.

(51) Int. Cl. A61K 35/76; A61P 37/04; C07K 5/113; C07H 21/04; C07K 7/06; C12N 7/01; A61K 39/395

(52) U.S. Cl. 424/158.1; 435/235.1; 424/199.1; 536/23.72; 530/328; 530/330; 530/329

(57) ABSTRACT

Disclosed are recombinant bacteriophage constructs and related heterologous peptide sequences for contraception in animals. The disclosed recombinant phage constructs bind to antibodies against gonadotropin releasing hormone (GnRH) and can be administered to an animal to generate an immune response against GnRH, including generating anti-GnRH antibodies. The disclosed recombinant phage may comprise an amino acid sequence of gonadotropin releasing hormone (GnRH), epitopic fragments, variants, or functional mimics thereof. Also disclosed are methods for making and selecting such recombinant phage constructs and compositions that comprise such constructs (e.g., compositions for inducing an immune response against GnRH including pharmaceutical or veterinary compositions used as vaccines). Also disclosed are recombinant polynucleotides comprising genomic nucleic acid of the recombinant phage constructs disclosed herein.

### CROSS-REFERENCE TO RELATED APPLICATIONS

The present application claims the benefit of priority under 35 U.S.C. §119(e) to U.S. provisional application No. 61/356,858, filed on Jun. 21, 2010, the content of which is incorporated herein by reference in its entirety.

### BACKGROUND

The present subject matter relates to the field of recombinant bacteriophage constructs and related heterologous peptide sequences for contraception in animals. In particular, the present subject matter relates to recombinant phage constructs that bind to antibodies against gonadotropin releasing hormone (GnRH) and can be administered to an animal to generate an immune response against GnRH, including generating anti-GnRH antibodies.

Gonadotropin releasing hormone (GnRH) is secreted by pituitary gland and acts as master reproductive hormone via regulation of the release of two major gonadotrophic hormones, luteinizing hormone (LH) and follicle-stimulating hormone (FSH). GnRH has been extensively studied for development of immunocontraceptive vaccines for various mammalian species, including pigs (Oonk et al., 1998; Zeng et al., 2001; Killian et al., 2006), rodents (Khan et al., 2008), deer (Miller et al., 2008), cats (Levy et al., 2004; Kutzler and Wood, 2006), dogs (Jung et al., 2005; Kutzler and Wood, 2006), and wild carnivores (Kutzler and Wood, 2006). GnRH vaccines stimulate antibody production to inactivate endogenous GnRH that in turn causes reduced release of gonadotrophic hormones leading to gonadal atrophy in adult animals or lack of development in sexually immature animals. This resulting regression or lack of development of the reproductive organs is referred to as immunological sterilization. Development of immunocontraceptive vaccines based on GnRH is challenging for several reasons: 1) GnRH is a small decapeptide with very low antigenicity and 2) it is naturally present in the body. Thus, it is recognized by the immune system as a “self” protein with no to very low antibody response following administration into an animal. To overcome these problems, GnRH has been conjugated to various antigenic molecules, for example, protein carriers (Kutzler and Wood, 2006) and T helper cell epitopes (Jung et al., 2005). Vaccinations with such constructs/molecules resulted in some levels of success in different species, but low immunogenicity remains a persistent difficulty. In addition, production of fusion proteins and synthetic peptides is costly and their shelf lifetime is limited.

Here, a novel type of GnRH vaccine is proposed that is based on phage-GnRH constructs or their structural or functional mimics. Phages are bacterial viruses that are used in the field as carrier proteins/adjuvants in preparation of vaccines for multiple applications (Zuercher et al., 2000; Manoutcharian et al., 2001; Frenkel et al., 2003; Fang et al., 2005; Wang et al., 2006; Yang et al., 2007; Eriksson et al., 2007; Eriksson et al., 2009; Houimel et al., 2009). Recombinant phage particles expressing foreign (non-phage) peptides, upon administration into an animal via different administration routes (subcutaneous, intramuscular, intraperitoneal, intranasal, oral, intravenous, intratumoral), are known to stimulate production of anti-phage antibodies as well as anti-foreign peptide (anti-peptide) antibodies (Minenkova et al., 1993). It has been also reported as well that more copies of the expressed peptide result in production of higher antipeptide antibody titers (Yip et al., 2001). Phage expressing foreign peptides can be selected from phage display libraries as well as constructed by the insertion of oligonucleotide sequences coding for the peptide of interest into a phage vector. For the first time, recombinant phage constructs that express GnRH peptide are proposed. These phage-GnRH express complete or truncated GnRH sequences, GnRH sequences with one or more amino acid substitutions, or mimics thereof on the phage surface. As such, the phage body may function as a protein carrier/adjuvant and multiple copies of GnRH peptides or their mimics will stimulate production of anti-GnRH antibodies. Different types of phage may be used to generate phage-GnRH antigenic constructs. In particular, phage-GnRH constructs based on landscape phage may be generated (Petrenko et al., 1996). In the landscape phage display libraries, foreign peptides are expressed in each pVIII copy over the phage surface. Therefore, insertion of the oligonucleotide sequence coding for GnRH peptide in gene VIII will result in expression of thousands of GnRH copies on phage surfaces significantly enhancing peptide antigenicity. In addition, recombinant phage preparations based on landscape phage are very thermostable. They are stable for more than six months at room temperature, more than six weeks at 63° C., and three days at 76° C. (Brigati and Petrenko, 2005) making landscape phage-based preparations very robust during shipping, storage, and operation.

### SUMMARY

Disclosed are recombinant bacteriophage constructs and related heterologous peptide sequences for contraception in animals. The disclosed recombinant phage constructs bind to antibodies against gonadotropin releasing hormone (GnRH) EHWSYGLRPG (SEQ ID NO: 1) and can be administered to an animal to generate an immune response against GnRH, including generating anti-GnRH antibodies. The disclosed recombinant phage may comprise an amino acid sequence of gonadotropin releasing hormone (GnRH), epitopic fragments, variants, or functional mimics thereof. Also disclosed are methods for making and selecting such recombinant phage constructs and compositions that comprise such constructs (e.g., compositions for inducing an immune response against GnRH including pharmaceutical or veterinary compositions used as vaccines). Also disclosed are recombinant polynucleotides comprising genomic nucleic acid of the recombinant phage constructs disclosed herein.

The heterologous peptide typically includes one or more epitopes of GnRH or functional mimics thereof. The heterologous may be at least 5, 6, 7, 8, 9, or 10 amino acids in length.

The heterologous peptide may include the amino acid sequence of GnRH EHWSYGLRPG (SEQ ID NO:1), epitopic fragments, variants, or functional mimics thereof. In some embodiments, the heterologous peptide comprises amino acid sequence EH. In other embodiments, the heterologous peptide comprises amino acid sequence EHWS (SEQ ID NO:2). In further embodiments, the heterologous peptide comprises amino acid sequence EWS. In even further embodiments, the heterologous peptide comprises amino acid sequence RPG. In still even further embodiments, the heterologous peptide comprises amino acid sequence EHWS (SEQ ID NO:2) and amino acid sequence RPG.

The heterologous peptide may comprise an amino acid sequence having sequence identity to any of the peptides disclosed herein (e.g., SEQ ID NOS:1-29). For, example, the heterologous peptide may comprise an amino acid sequence having at least about 20%, 30%, 40%, 50%, 60%, 70%, 80%, or 90% sequence identity to any of SEQ ID NOS:1-29.

The heterologous peptide is inserted within a protein of the bacteriophage. For example, the heterologous peptide may be inserted within a pVIII protein of the bacteriophage. In some embodiment, the filamentous bacteriophage has no more than a single gene 8. The bacteriophage may be a landscape bacteriophage.

Also disclosed are immunogenic compositions that include the disclosed phage and a suitable excipient, carrier or diluent. The compositions further may comprise an adjuvant. The compositions may be formulated as pharmaceutical or veterinary compositions, for example, for administering as a vaccine.

The immunogenic compositions may be administered to an animal in methods for generating an immune response against GnRH, which may include B-cell responses (e.g., antibody responses) and/or T-cell responses. The immunogenic compositions may be administered to an animal in methods for immunizing the animal against conception or for reducing fertility in the animal.

### BRIEF DESCRIPTION OF THE FIGURES

FIG. 1. Titers of anti-peptide antibody in mice injected with EHPAGMTGD (SEQ ID NO:3) phage-peptide construct. Mice were bled three times with the intervals of 3 to 4 weeks.

FIG. 2. Cloning site of phage vector f8-6.

FIG. 3. Phage-GnRH peptide constructs obtained via cloning in f8-6 phage vector (underlined heterologous peptide amino acids—GnRH epitopes; non-underlined heterologous peptide amino acids—amino acid substitutions or spacer amino acids. Phage amino acids (A.A.) also are shown).

FIG. 4. Anti-peptide antibody titer in serum collected from a dog immunized with EHWSYGLDPA (SEQ ID NO:6) phage-peptide construct.

FIG. 5. Section from left testicle showing degenerative changes within the seminiferous tubules characterized by cytoplasmic vacuolization, decreased germinal epithelial thickness and desquamation of cells. Focal inflammatory cells are present in the interstium to the left of the photo.

FIG. 6. Section showing degeneration of seminiferous tubules showing vacuolization with almost total loss of germinal cells.

FIG. 7. Section showing severe vacuolization of tubules and loss of germinal epithelium.

FIG. 8. Epididymis. There are no normal sperm within the epididymal tubules. This is an example showing the epididymal tubules with some focal inflammation in the interstitial connective tissue (A) and proteinaceous and cellular debris in other sections (B). (C) shows the cellular debris within epididymis at a higher power.

### DETAILED DESCRIPTION

The disclosed subject matter is further described below.

Unless otherwise specified or indicated by context, the terms “a”, “an”, and “the” mean “one or more.” For example, “a peptide” should be interpreted to mean “one or more peptides” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

The terms “subject” and “patient” may be used interchangeably herein. A patient or subject may refer to a non-human patient or subject at risk for conception. Non-human patients (i.e., animals) may include mammals.

The recombinant phage and heterologous peptides disclosed herein bind specifically to anti-GnRH antibodies. Furthermore, the recombinant phage and heterologous peptides disclosed herein contemplated herein may be utilized in immunogenic compositions or vaccines for eliciting antibodies that bind specifically to GnRH. In this regard, the terms “binds specifically” and “bind specifically” refer to that interaction between the recombinant phage and heterologous peptides and anti-GnRH antibodies. The interaction is dependent upon the presence of a particular structure of the recombinant phage or heterologous peptides, e.g., the antigenic determinant or epitope present on the recombinant phage or heterologous peptides, recognized by the anti-GnRH antibody or binding molecule. For example, if the anti-GnRII antibody is specific for epitope “A,” the presence of a recombinant phage or heterologous peptides comprising the epitope A, or the presence of free unlabeled A, in a reaction containing free labeled A and the anti-GnRH antibody will reduce the amount of labeled A that binds to the anti-GnRH antibody.

The heterologous peptides disclosed herein may be described via their “amino acid sequence.” As used herein, the term “amino acid sequence” refers to amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (H is or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of pyroglutamic acid, homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methyl isoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The presently disclosed heterologous peptides may be synthetic. As used herein, “synthetic peptide” refers to a peptide which has an amino acid sequence which is not a native sequence or is not in its native context and which confers on phage displaying it the ability to bind or preferentially bind to a particular cell population. By “not in its native context” is intended that the peptide is substantially or essentially free of amino acid sequences that naturally flank the amino acid sequence of the peptide in the native protein which comprises the amino acid sequence of the peptide. For example, a synthetic peptide which is not in its native context may be flanked at either or both ends by no more than 50, 40, 30, 20, 10, 9, 8, 7, 6, 5, 4, 3, 2, or 1 amino acid(s) found in the native protein.

The terms “peptide” and “polypeptide” (and/or “protein”) may be used interchangeably herein. However, generally a peptide is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≧100 amino acids (Garrett & Grisham, Biochemistry, 2″ edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues. A “protein” generally refers to a polypeptide (or peptide), which optionally may be further modified to include non-amino acid moieties and which exhibits a biological function.

The peptides and polypeptides contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

“Sequence identity” refers to sequence similarity or, interchangeably, sequence homology, between two or more polypeptide sequences or two or more polynucleotide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant,” “mutant,” or “derivative” of a particular polypeptide sequence is defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” or a “derivative” may have substantially the same functional activity as a reference polypeptide. For example, a variant or derivative of GnRH may have GnRH functional activity and be capable of binding to the GnRH receptor or may be a structural mimic capable of inducing anti-GnRH antibodies.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed below).

Percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

Preferably, the recombinant phage and heterologous peptides disclosed herein selectively bind to anti-GnRH antibodies relative to control antibodies (e.g., preimmune serum). The recombinant phage and heterologous peptides disclosed herein may exhibit at least two-fold, three-fold, four-fold, five-fold, six-fold, seven-fold, ten-fold, twenty-fold, thirty-fold or more increased binding affinity for anti-GnRH antibodies relative to control antibodies. Recombinant phage and heterologous peptides that exhibit such binding characteristics are said to exhibit preferential binding to anti-GnRH antibodies. Recombinant phage and heterologous peptides that do not exhibit at least a two-fold increased binding affinity for anti-GnRH antibodies relative to control antibodies are simply said to bind to anti-GnRH.

Also contemplated herein are “nucleic acid sequences” that encode the disclosed phage proteins (e.g., genomic phage nucleic acid) and heterologous peptide. As used herein, the term “nucleic acid sequence” refers to a nucleotide, oligonucleotide, polynucleotide, or any fragment thereof. As used herein, the term “polynucleotide” refers to a nucleotide polymer. A polynucleotide may encode a peptide or polypeptide as disclosed herein. A polynucleotide may be operably linked to a heterologous promoter sequence as a recombinant polynucleotide. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. A recombinant polynucleotide comprising a polynucleotide operably linked to a promoter sequence may be present in a vector (e.g., a plasmid) which may be utilized to transform a host cell (e.g., where the vector further includes a selectable marker). Accordingly, the disclosed peptides and polypeptides may be expressed in a host cell via an encoding nucleic acid sequence.

The heterologous peptides disclosed herein may be expressed via vectors that include viral, bacterial, or other vectors. Preferably, the heterologous peptides are expressed via a recombinant filamentous bacteriophage (i.e., Ff class bacteriophages). Bacteriophages (or phages) are viruses that infect bacteria. They consist of an outer protein capsid enclosing genetic material (single stranded circular DNA). Filamentous phages (Ff class) are long (˜1 μm) and thin (˜7 nm) particles. Such particles can be genetically re-engineered and utilized as carriers for immunogenic peptides, which are displayed on phage surfaces as fusion molecules to phage coat proteins. (See Minenkova et al., Gen. 1993:85-88; and Yip et al., Immunol. Lett. 2001 Dec. 3; 79(3):197-202). To create fusion peptides, a foreign oligonucleotide usually is inserted into the phage minor coat protein gene 3 or the phage major coat protein gene 8. Inserts in gene 3 produce fusions with a maximum of 5 copies of protein III (pIII). Inserts in gene 8 produce multiple fusion peptides, the number of which depends on the phage vector design. Usually, protein VIII (pVIII) vectors contain two copies of gene 8, one of which encodes the wild-type protein, and the other encodes a fusion protein. Such vector design results in irregular phage surface architecture that contains a variable number (from 15 to 300) of fusion peptides separated by wild-type phage proteins. However, by using a bacteriophage vector that has only one copy of gene 8, all copies of the major coat protein VIII are modified with the fusion peptide. (See Petrenko et al., Protein Eng. 1996 September; 9(9):797-801). Such a bacteriophage may otherwise be referred to as a “landscape phage” in view of the dramatic change in surface structure of the phage caused by the >1000 copies of the heterologous peptide present in a dense, repeating pattern on the phage's tubular capsid. In landscape phage, foreign peptides are expressed in each copy of the phage major coat protein VIII, resulting in a surface density of as many as 4000 foreign peptide copies per phage particle. Most importantly, high density epitopes in the landscape phage are presented in a highly-organized manner and are properly spaced for binding to B cell receptors. Such repetitive highly-organized epitope patterns usually permit a cross-linking activation of B-cell receptors, which provides robust, long-lasting immune responses. (See Bachmann et al., Annu. Rev. Immunol. 1997; 15:235-70; and Fehr et al., Proc. Natl. Acad. Sci. USA 1998 Aug. 4; 95(149477-81). Additionally, phage are able to stimulate strong T helper cell responses. (See Gaubin et al., DNA Cell Biol. 2003 January; 22(1):11-18; Hashemi et al., J. Virol. Methods 2010 February; 163(2):440-444; Ulivieri et al., Immunol. Lett. 2008 Aug. 15; 119(1-2):62-70; and Wan et al., Eur. J. Immunol. 2005 July; 35(7:2041-50). Phage particulate nature, size and shape further appeal for its strong/long-lasting immunogenic potentials. Filamentous phage has been shown to naturally stimulate both B and T helper cell responses without adjuvants. (See De Berardinis et al., Expert Rev. Vaccines 2004 December; 3(6):673-9; and Manoutcharian et al., Curr. Pharm. Biotechnol. 2001 September; 2(3):217-23). Although they can not infect animal cells, landscape phage may be inactivated prior to subsequent use in an immunogenic or vaccine composition.

Phages, as bacterial viruses, can be easily obtained in large quantities from bacterial cultures, which makes the cost of phage preparations much lower than the cost of peptides vectored in mammalian viruses or the cost of production of synthetic peptides. Importantly, landscape phage preparations are very thermostable. They resist degradation and retain antigenicity for more than six months at room temperature, more than six weeks at 63° C., and three days at 76° C. (see Brigati et al., Anal. Bioanal. Chem. 2005 July; 282(6):1346-50), making landscape phage-based preparations ideally suited for shipping, storage, and delivery in field conditions without requiring refrigeration. Examples of phage-based (non-landscape phage) vaccines reported in the literature include preparations for treatment of melanoma (Eriksson et al., Cancer Immunol. Immunother. 2007 May; 56(5):677-87; and Eriksson et al., J. Immunol. 2009 Mar. 1; 182(5):3105-11), HIV (see De Berardinis et al., Curr. HIV Res. 2003 October; 1(4):441-6), Alzheimer's disease (see Frenkel et al. Vaccine 2003 Mar. 7; 21(11-12):1060-5), candidiasis (see Wang et al., Vaccine 2006 Aug. 28; 24(35-36):6065-73; and Yang et al., Mycoses 2007 May; 50(3):165-71), and rabies (see Houimel et al., Vaccine 2009 Jul. 23; 27(34):4648-55). Furthermore, recombinant phages displaying decapeptides of follicle-stimulating hormone receptor were shown to impair fertility in mice and inhibit ovulation rates in ewes (see Abdennebi et al., J. Mol. Endocrinol. 1999 April; 22(2):151-9) and to induce infertility in adult male bonnet monkeys (see Rao et al., Reprod. Biomed. Online 2004 April; 8(4):385-91), suggesting the potential use of phage-based vaccines for immunocontraception. These vaccines were shown to stimulate anti-peptide responses with only 5 to 300 peptide copies (irregularly spaced) per phage particle, stimulating both systemic and mucosal immunity. Filamentous phage preparations based on fd phage (same as landscape phage) have been used experimentally in humans with the approval of the FDA and with no apparent side effects (see Krag et al., Cancer Res. 2006 Aug. 1; 66(15):7724-33), indicating their safety.

Recombinant phage particles displaying fusion peptides can be obtained via cloning of oligonucleotides encoding for the fusion peptides in phage display vectors. Alternatively, phage clones displaying desired peptides or their structural/functional mimics can be selected from phage display libraries.

The presently disclosed peptides, polypeptides, landscape phage, or vectors may be isolated or substantially purified. The terms “isolated” or “substantially purified” refers to peptides, polypeptides, landscape phage, or vectors that have been removed from their environment and have been isolated or separated, and are at least 60% free, preferably at least 75% free, and more preferably at least 90% free, and most preferably at least 95% free from other components with which they were naturally associated.

Also disclosed are heterologous peptides identified by the phage display method disclosed herein, and preferably include peptides that bind to anti-GnRH antibodies and that can induce an anti-GnRH immune response when administered to an animal either alone or as part of a recombinant phage or other vector. Peptides identified herein include peptides having the amino acid sequence or motifs of SEQ ID NOS:1-29. Also disclosed are polypeptides comprising the amino acid sequence or motifs of any of SEQ ID NOS:1-29, polynucleotides encodings such polypeptides, recombinant polynucleotides comprising such polynucleotides, expression vectors, and methods for expressing the encoded polypeptide.

The peptides disclosed herein may be fused or conjugated to one or more other peptides or non-peptide moieties (e.g., in order to provide an antigen). For example, a fusion polypeptide as contemplated herein may include a fusion of any of the peptides or motifs of SEQ ID NO: 1-29 and one or more other immunogenic peptides. The peptides disclosed herein may be present in a polypeptide (e.g., where the polypeptide comprises one or more copies of the amino acid sequence of the peptide, optionally in tandem). The disclosed peptides may be modified to enhance immunogenicity. For example, the peptides disclosed herein may be conjugated to one or more carrier proteins.

The disclosed methods may include inducing an immune response against GnRH. In some embodiments, the methods include inducing polyclonal antibodies against GnRH by administering to an animal an immunogenic composition that includes a recombinant phage comprising a heterologous peptide as disclosed herein. The animal may be a non-human mammal. The induced polyclonal antibodies may include anti-GnRH antibodies. The methods disclosed herein also may include preventing conception by administering to the animal an immunogenic composition that includes recombinant phage comprising a heterologous peptide as disclosed herein. For example, an animal (e.g., a non-human mammal) may be protected against conception by administering to the animal a composition that includes a recombinant phage comprising a heterologous peptide as disclosed herein together with a suitable excipient.

The disclosed compositions may be administered as immunogenic compositions or vaccines utilizing a selected “prime-boost vaccination regimen.” As used herein, a “prime-boost vaccination regimen” refers to a regimen in which a subject is administered a first composition one or more times (e.g., one time or two or three times with about 2, 3, or 4 weeks between administrations) and then after a determined period of time after having administered the first composition (e.g., about 2 weeks, about 4 weeks, about 2 months, about 3 months, about 4 months, about 5 months, about 6 months, or longer), the subject is administered a second composition. The second composition may also be administered more than once, with at least 2, 3, or 4 weeks between administrations. The first and second compositions may be the same or different.

Also disclosed are immunogenic compositions and vaccines for performing the disclosed methods. The immunogenic or vaccine compositions may comprise a recombinant phage comprising a heterologous peptide as disclosed herein or another vector that expresses the disclosed heterologous peptides. The disclosed immunogenic or vaccine compositions may be monovalent or polyvalent. For example, the immunogenic compositions may include one or more landscape phage that express one or more different heterologous peptides. The immunogenic compositions also may include a suitable excipient, carrier, or diluent.

Suitable peptides for the immunogenic compositions (or for expression by vectors of the immunogenic compositions) may include one or more polypeptides comprising the amino acid sequence of a peptide as disclosed herein, for example one or more polypeptides comprising the amino acid sequence or motifs of any of SEQ ID NOS:1-29. In some embodiments, the immunogenic compositions may include two or more polypeptides (or two or more vectors that express two or more polypeptides) where each polypeptide of the two or more polypeptides comprises the amino acid sequence or motifs of any of SEQ ID NOS:1-29. The immunogenic compositions may include an isolated peptide at a concentration sufficient to induce an immunogenic response against GnRH (e.g.; via antibody induction, a T-cell response, or both), or the immunogenic compositions may include one or more vectors that express the polypeptide or peptide at a concentration sufficient to induce an immunogenic response against GnRH (e.g., via antibody induction, a T-cell response, or both).

The “immunogenic compositions” and “vaccines” disclosed herein are capable of stimulating an immune response in an animal inoculated with the immunogenic composition or vaccine. An immune response may include induction of antibodies, induction of a T-cell response, or both. Herein, the term “prevention” when used in reference to an immunogenic composition or vaccine may refer to the partial or complete prevention against conception via an immune response induced by the immunogenic composition or vaccine.

An “an immunogenic composition comprising a given peptide or polypeptide” refers to a composition containing the given peptide or polypeptide. The composition may comprise a dry formulation or an aqueous solution. An “immunogenic peptide or polypeptide” is an antigen which is capable of eliciting an immune response when introduced into an animal, for example, a non-human mammal.

The methods disclosed herein may include administering an immunogenic composition or a vaccine to an animal. An “animal,” as used herein, may include a non-human mammal.

In some embodiments, the disclosed peptides or polypeptides may be expressed by a vector other than a landscape phage, for example animal virus vectors or bacterial vectors (e.g., as included as part of an immunogenic composition, vaccine, or bait composition). As used herein, an “animal viral vector” refers to recombinant animal virus nucleic acid that has been engineered to express a heterologous polypeptide. The recombinant animal virus nucleic acid typically includes cis-acting elements for expression of the heterologous polypeptide. The recombinant animal virus nucleic acid typically is capable of being packaged into a helper virus that is capable of infecting a host cell. For example, the recombinant animal virus nucleic acid may include cis-acting elements for packaging. Typically, the animal viral vector is not replication competent or is attenuated. An “attenuated recombinant virus” refers to a virus that has been genetically altered by modern molecular biological methods (e.g., restriction endonuclease and ligase treatment, and rendered less virulent than wild type), typically by deletion of specific genes. For example, the recombinant animal virus nucleic acid may lack a gene essential for the efficient production or essential for the production of infectious virus. Suitable animal viral vectors for expressing the peptides and polypeptides disclosed herein may include, but are not limited to adenovirus vectors, Sendai virus vectors, and measles virus vectors. Recombinant attenuated bacteria also may be utilized as vectors in the pharmaceutical compositions and vaccines disclosed herein (e.g., recombinant attenuated Shigella, Salmonella, Listeria, or Yersinia). Recombinant bacterial vaccine vectors are described in Daudel et al., “Use of attenuated bacteria as delivery vectors for DNA vaccines,” Expert Review of Vaccines, Volume 6, Number 1, February 2007, pp. 97-110(14); Shata et al., “Recent advances with recombinant bacterial vaccine vectors,” Molec. Med. Today (2000), Volume 6, Issue 2, 1 Feb. 2000, pages 66-71; Clare & Dougan, “Live Recombinant Bacterial Vaccines,” Novel Vaccination Strategies, Apr. 16, 2004 (Editor Stefan H. E. Kaufman); Gentschev et al., “Recombinant Attenuated Bacteria for the Delivery of Subunit Vaccines,” Vaccine, Volume 19, Issues 17-19, 21 Mar. 2001, Pages 2621-2628; Garmory et al., “The use of live attenuated bacteria as a delivery system for heterologous antigens,” J. Drug Target. 2003; 11(8-10):471-9; U.S. Pat. No. 6,383,496; and U.S. Pat. No. 6,923,958 (which all are incorporated by reference herein in their entireties). Preferably, the vector is species-specific, whereby the vector selectively infects a target species of animal or the vector selectively expresses an encoded heterologous peptide in the target species of animal after infecting the animal.

The immunogenic compositions or vaccines may be formulated for delivery in any suitable manner. For example, the immunogenic compositions or vaccines may be formulated for at least one of oral delivery, intranasal delivery, intramuscular delivery, subdermal delivery, subcutaneous delivery, intravenous delivery, and intraperitoneal delivery. The immunogenic compositions or vaccines can be administered using a variety of methods including intranasal and/or parenteral (e.g., intramuscular) administration. In some embodiments of the methods, the immunogenic composition or vaccine is administered intramuscularly one or more times at suitable intervals (e.g., at intervals of 2-4 weeks), followed by administration of the immunogenic composition or vaccine at least once intramuscularly or intranasally after a suitable time period (e.g., 2-4 weeks after the last parenteral administration of vaccine). The immunogenic compositions or vaccines may be administered to an animal of either sex. In some embodiments, the animal is female.

The present immunogenic composition and vaccines may be formulated with a pharmaceutically or veterinarily acceptable excipient, carrier, or diluent. The forms suitable for injectable commonly include sterile aqueous solutions (where water soluble) or dispersions and sterile powders for the extemporaneous preparation of sterile injectable solutions or dispersion. The formulation should desirably be sterile and fluid to the extent that easy syringability exists. The dosage form should be stable under the conditions of manufacture and storage and typically is preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. The proper fluidity of the solution can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal (sodium ethylmercuri-thiosalicylate), deomycin, gentamicin and the like. In many cases it may be preferable to include isotonic agents, for example, sugars or sodium chloride. Prolonged absorption of the injectable compositions, if desired, can be brought about by the use in the compositions of agents delaying absorption, for example, aluminum monostearate and gelatin.

The present immunogenic composition or vaccines may include an adjuvant. The term “adjuvant” refers to a compound or mixture that is present in an immunogenic composition or vaccine and enhances the immune response to an antigen present in the immunogenic composition or vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL1005), aluminum phosphates (e.g., AIPO₄), R-848 (a Th1-like adjuvant), imiquimod, PAM3CYS, poly (I:C), loxoribine, potentially useful human adjuvants such as BCG (bacille Calmette-Guerin) and Corynebacterium parvum, CpG oligodeoxynucleotides (ODN), cholera toxin derived antigens (e.g., CTAI-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

It is generally advantageous to formulate the present compositions in dosage unit form for ease of administration and uniformity of dosage. “Dosage unit form” as used herein refers to physically discrete units suited as unitary dosages for the animal subjects to the treated; each unit containing a predetermined quantity of the active material calculated to produce the desired therapeutic effect in association with the required pharmaceutical carrier. The specification for the dosage unit forms are dictated by and depend on among other factors (a) the unique characteristics of the active material and the particular therapeutic effect to be achieved; (b) the limitations inherent in the art of compounding such active material for the treatment of disease; and (c) the manner of intended administration of the dosage unit form. In some embodiments, a dose of the immunogenic composition or vaccine includes at least about 10 micrograms (preferably 100 micrograms) of one or more isolated polypeptides or peptides as disclosed herein.

Sterile injectable solutions may be prepared by incorporating the isolated polypeptide or peptide in the desired amount in an appropriate solvent with various of the other ingredients enumerated above, as required, followed by filtered sterilization. Generally, dispersions can be prepared by incorporating the various active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient (i.e., lyophilized form of the active ingredient) plus any additional desired ingredient from a previously sterile-filtered solution thereof.

It also may be advantageous to add a stabilizer to the present compositions. Suitable stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin). If desired, the formulation may be buffered by methods known in the art, using reagents such as alkali metal phosphates, e.g., sodium hydrogen phosphate, sodium dihydrogen phosphate, potassium hydrogen phosphate and/or potassium dihydrogen phosphate. Other solvents, such as ethanol or propylene glycol, can be used to increase solubility of ingredients in the vaccine formulation and/or the stability of the solution. Further additives which can be used in the present formulation include conventional antioxidants and conventional chelating agents, such as ethylenediamine tetraacetic acid (EDTA).

Also disclosed herein are isolated antisera, antibodies, or other binding molecules that bind specifically to the peptides disclosed herein. For example, the antisera, antibodies, or other binding molecules, may include an isolated antibody that binds specifically to a polypeptide consisting of an amino acid sequence or motif of any of SEQ ID NOS:1-29. Preferably, the antisera, antibodies, or other binding molecules disclosed herein also bind specifically to GnRH. The isolated antibody or binding molecule may be of any suitable isotype (e.g., IgG, IgM, IgE, IgD, IgA, and mixtures thereof). The antibodies may be polyclonal or monoclonal. The term “antibody or other binding molecule” refers to intact immunoglobulin molecules as well as to fragments thereof, such as Fab, F(ab′)₂, and Fv fragments, which are capable of binding an epitopic determinant. The antibodies or other binding molecules may be naturally occurring or synthetic (e.g., scFv). Other binding molecules may include antibody fragments (e.g., Fab fragments), coupled antibodies, and coupled antibody fragments. Antibodies or other binding molecules that bind the presently disclosed peptides and polypeptides can be induced or elicited using the intact peptide or a polypeptide comprising the intact peptide as an immunizing antigen. The polypeptide or oligopeptide used to immunize an animal can be derived from the translation of DNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide may then be used to immunize the animal. The antibodies or other specific binding molecules may be conjugated to a suitable therapeutic agent (e.g., a toxin) or a label. The antibodies may be modified for use in therapeutic or diagnostic methods.

Also disclosed herein are kits. The kits may include one or more components for performing the methods disclosed herein. For example, the kits may include one or more of the immunogenic compositions or vaccines for immunizing or vaccinating an animal. The components of the disclosed kits may be provided in any suitable form (e.g., liquid form or lyophilized form). Kits further may include solvents for resuspending or dissolving a lyophilized protein.

### ILLUSTRATIVE EMBODIMENTS

The following embodiments are illustrative and are not intended to limit the claimed subject matter.

### Embodiment 1

A method for obtaining an antigen for generating an immune response against gonadotropin releasing hormone (GnRH), the method comprising: (a) contacting a phage library with anti-GnRH antibodies; and (b) selecting phage from the library that bind specifically to the anti-GnRH antibodies.

### Embodiment 2

The method of embodiment 1, wherein the phage library is first contacted with control antibodies and phage that bind to the control antibodies are removed from the phage library.

### Embodiment 3

An isolated recombinant filamentous phage comprising a heterologous peptide, wherein the phage binds to an antibody against gonadotropin releasing hormone.

### Embodiment 4

The phage of embodiment 3, wherein the heterologous peptide is at least about five amino acids in length.

### Embodiment 5

The phage of any of the preceding embodiments, wherein the heterologous peptide is at least about eight amino acids in length.

### Embodiment 6

The phage of any of the preceding embodiments, wherein the heterologous peptide is at least about nine amino acids in length.

### Embodiment 7

The phage of any of the preceding embodiments, wherein the heterologous peptide is at least about ten amino acids in length.

### Embodiment 8

The phage of any of the preceding embodiments, wherein the heterologous peptide comprises amino acid sequence EH.

### Embodiment 9

The phage of any of the preceding embodiments, wherein the heterologous peptide comprises amino acid sequence EHWS (SEQ ID NO:2).

### Embodiment 10

The phage of any of the preceding embodiments, wherein the heterologous peptide comprises amino acid sequence EWS.

### Embodiment 11

The phage of any of the preceding embodiments, wherein the heterologous peptide comprises amino acid sequence RPG.

### Embodiment 12

The phage of any of the preceding embodiments, wherein the heterologous peptide comprises amino acid sequence EHWS (SEQ ID NO:2) and amino acid sequence RPG.

### Embodiment 13

The phage of claim 3, wherein the heterologous peptide comprises an amino acid sequence having at least about 50% sequence identity to EHWSYGLRPG (SEQ ID NO:1).

### Embodiment 14

The phage of claim 3, wherein the heterologous peptide comprises an amino acid sequence having at least about 50% sequence identity to EHPAGMTGD (SEQ ID NO:3).

### Embodiment 15

The phage of claim 3, wherein the heterologous peptide comprises an amino acid sequence having at least about 50% sequence identity to EHWSYGLDPA (SEQ ID NO:6).

### Embodiment 16

The phage of claim 3, wherein the heterologous peptide is inserted within a pVIII protein of the bacteriophage.

### Embodiment 17

The phage of embodiment 16, wherein the filamentous bacteriophage has no more than a single gene 8.

### Embodiment 18

An immunogenic composition comprising: (a) the phage of embodiment 3; and (b) a suitable excipient, carrier, or diluent.

### Embodiment 19

The composition of embodiment 15, further comprising an adjuvant.

### Embodiment 20

A method for producing antibodies that bind to GnRH, the method comprising administering the immunogenic composition of embodiment 18 to an animal.

### Embodiment 21

A method for immunizing an animal against conception, the method comprising administering the immunogenic composition of embodiment 18 to the animal.

### Embodiment 22

An isolated polynucleotide comprising genomic nucleic acid of the recombinant bacteriophage of embodiment 3.

### Embodiment 23

An isolated peptide comprising an amino acid sequence selected from a group consisting of SEQ ID NOS:1-29.

### EXAMPLES

The following Examples are illustrative and are not intended to limit the scope of the claimed subject matter.

Phage-GnRH Constructs, their Mimics, Antigenic Compositions, and Sequences for Immunocontraception of Animals and Other Applications

To generate phage-GnRH constructs, two independent approaches were explored: 1) a phage display library was selected against anti-GnRH antibodies to identify phage carrying GnRH peptide or its mimics, and 2) oligonucleotide sequences encoding for exact or rationally modified GnRH peptide were inserted in a phage vector via standard cloning techniques.

(1) Phage-GnRH constructs obtained via selection from a phage display library: To identify phage carrying GnRH peptide or its mimics, the f8/9 landscape phage display library was selected on anti-GnRH antibodies. Commercially available antibodies (rabbit polyclonal, Abcam Inc., Cambridge, Mass.) were used in the phage selection experiments. To identify phage that specifically recognize and bind to the antibodies, f8/9 phage display library was reacted with the antibodies in three consecutive selection rounds. After the final round, phage DNAs were sequenced and translated into peptides. This resulted in identification of several phage clones displaying peptides with partial homology to GnRH peptide EIIWSYGLRPG (SEQ ID NO:1), including EHPAGMTGD (SEQ ID NO:3), EWSSSSTDT (SEQ ID NO:4) and ATDTRPGTE (SEQ ID NO:5), which contain EHWS (SEQ ID NO:3) and RPG epitopes known to bind to the GnRH receptor.

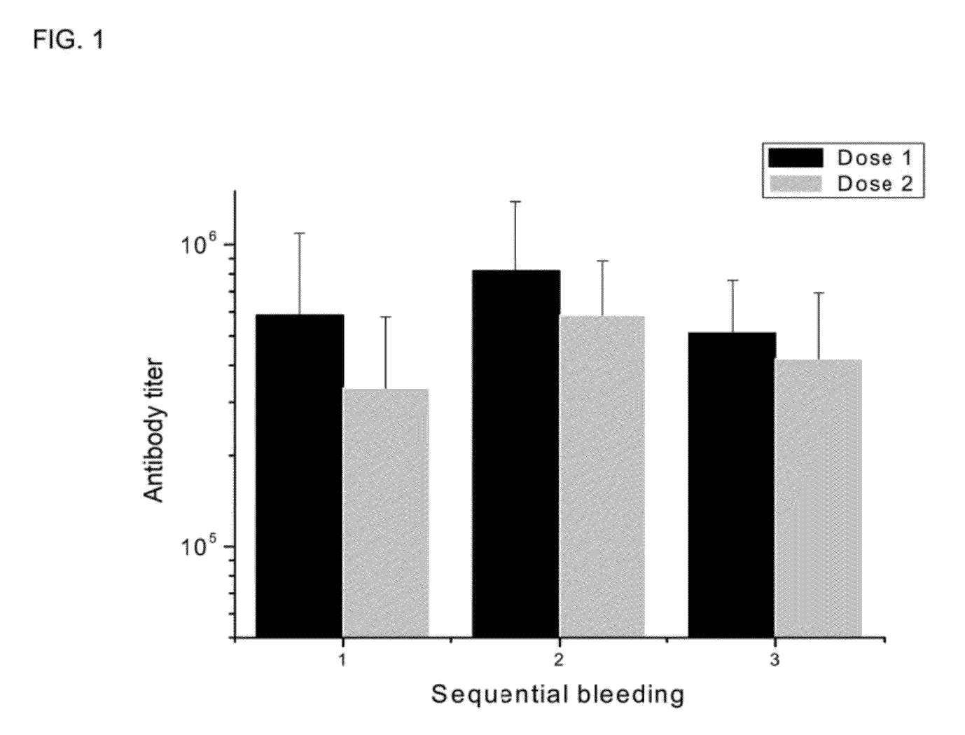

To confirm contraceptive properties of the phage-peptide constructs identified as above, one of the constructs, EHPAGMTGD (SEQ ID NO:3), was tested for reduction of pup numbers in fertility trials in mice. The following groups were established: (a) negative control group of mice injected with PBS; (b) carrier phage control group of mice injected with vector phage, and (c) test groups of mice injected with two different doses (10 μg or 15 μg) of the phage-peptide construct. Each mouse in all groups received the primary injection followed by a single boost injection four weeks later. Serum samples were collected prior to immunization and biweekly thereafter. The samples collected from the test groups were assayed for anti-peptide antibodies in ELISA format. It was shown (FIG. 1) that immunization of mice with EHPAGMTGD (SEQ ID NO:3) phage-peptide construct stimulated production of anti-peptide antibodies at high titers.

For the following fertility trials, immunized female and male mice were paired for at least two weeks and pups were counted. Table 1 contains the 95% confidence intervals for the mean number of pups per mouse for each treatment group. The data were analyzed using the GENMOD (generalized linear model) procedure in SAS. If the number of pups was summarized for more than one mother then their results were reported as half the aggregated number of pups. For example, in the PBS control groups two mothers were recorded as having a combined number of pups of 21. Because there was no way of telling the individual numbers for each mother, they were each recorded as 11.5 (21/2=11.5). After the generalized linear model was fitted, each of the phage dose groups were compared separately to the PBS controls and phage vector controls, using linear contrasts. Based on this GENMOD analysis, both dose groups had significantly lower number of pups as compared to the control groups (p-values=0.045 and 0.01, dose 1 and 2 dose, respectively). The estimated mean number of pups/mother for the EHPAGMTGD (SEQ ID NO:3) combined dose group is 9.1 (95% CI: (7.41, 11.18)) as compared to PBS controls 13.50 (11.18, 16.30). Note: when comparing all controls to the combined EHPAGMTGD (SEQ ID NO:3) (referred to as “EH” in the table) phage group (dose 1 and 2), the p-value=0.005.

TABLE 1
The mean pups/mouse and 95% confidence intervals for each
treatment group

| | Estimated mean | 95% Confidence Interval | |
|---|---|---|---|
| Group | pups/mouse | LowerCI | UpperCI |
| PBS Control (n = 8) | 13.50 | 11.18 | 16.30 |
| EH phage, dose1 (n = 5) | 9.6 | 7.23 | 12.74 |
| EH phage, dose 2 (n = 5) | 8.6 | 6.38 | 11.60 |
| EH phage 1 & 2 (n = 10) | 9.1 | 7.41 | 11.18 |

Conclusions: 1) immunization of mice with EHPAGMTGD (SEQ ID NO:3) phage-peptide construct stimulated production of anti-peptide antibodies at high titers, and 2) immunization of mice with EHPAGMTGD (SEQ ID NO:3) phage-peptide construct led to significant reduction in the numbers of pups born in fertility trials in very prolific outbred CDI mouse strain.

(2) Phage-Gnrh Peptide Constructs Obtained Via Cloning in a Phase Vector: Based on the immunocontraceptive role of individual amino acids in the GnRH sequence, knowledge about GnRH epitopes which bind to GnRH receptor, and properties of the landscape phage, multiple phage-GnRH peptide constructs of different length and composition were designed and constructed.

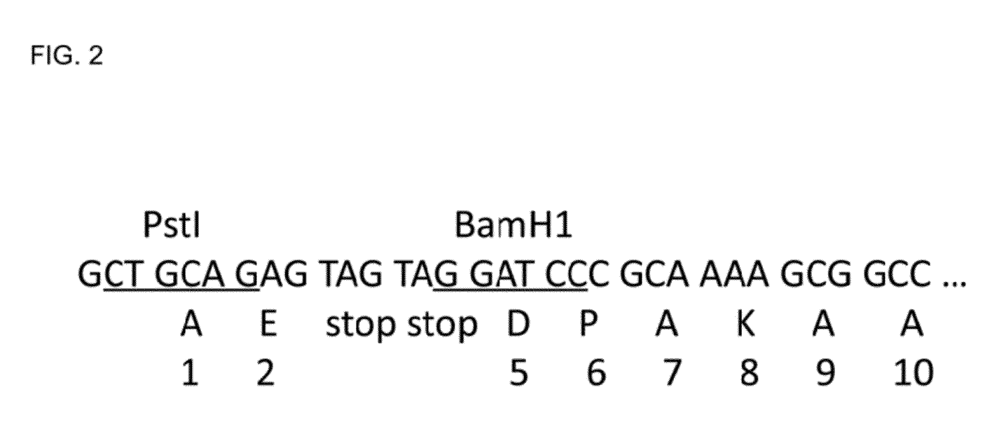

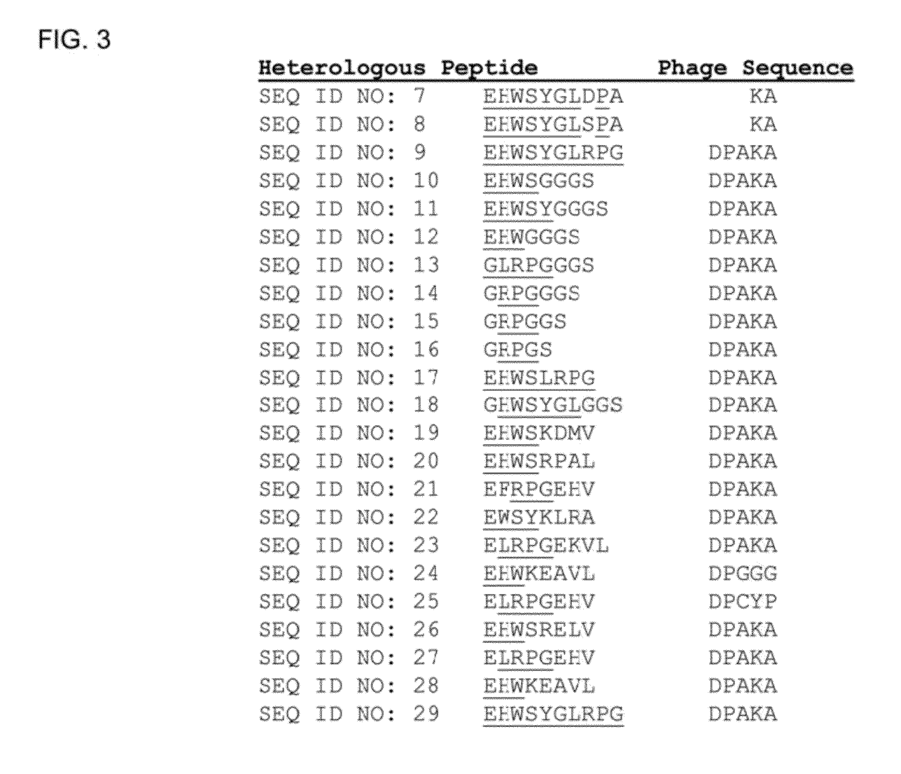

The constructs were generated via cloning of oligonucleotides encoding for exact or rationally modified GnRH sequence into f8-6 phage vector (FIG. 2). Clones containing new DNA inserts were verified by DNA sequencing. List of the clones generated as described above is shown in FIG. 3.

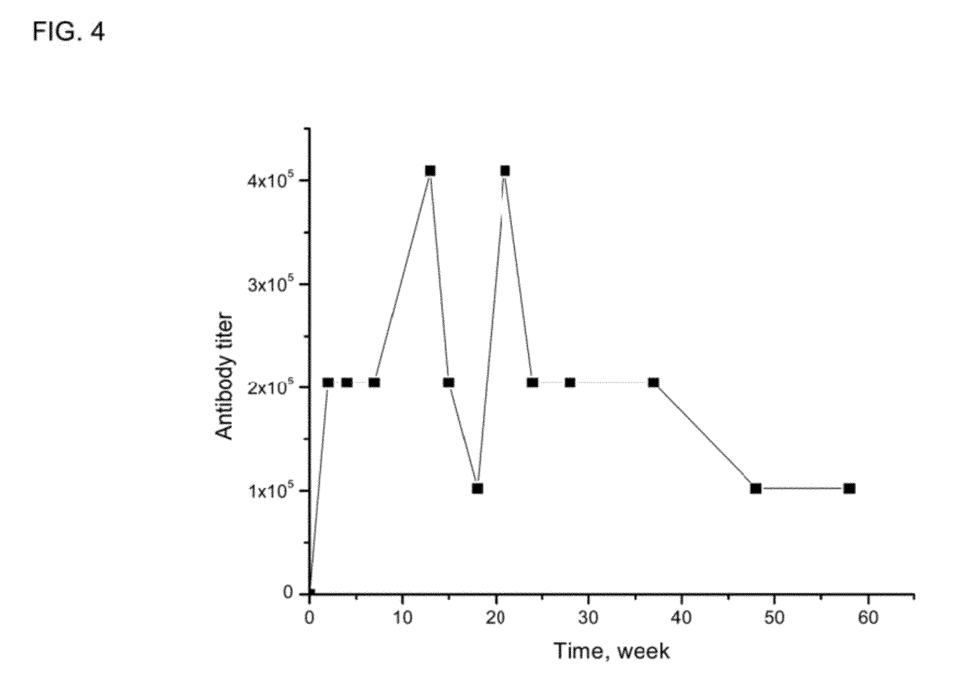

One of the phage-GnRH constructs obtained via cloning, EHWSYGLDPA (SEQ ID NO:6), was tested for suppression of reproductive functions in a one-year old male dog. The dog received a primary immunization (3.5×10¹¹ cfu phage) as well as three boosts at the doses ranging from 0.2×10¹¹ cfu to 1.3×10¹¹ cfu that took place from three to eight weeks apart. Serum samples were collected and characterized as to the presence of anti-peptide antibodies by ELISA. The phage injections were shown to induce production of high levels of serum IgG antibodies that persisted for at least 15 months (FIG. 4).

Additionally, testicular widths in this dog measured by a caliper were decreased when compared to the pre-immunization testicular size. Ultrasound examination of the dog's reproductive organs revealed that his testicles and prostate were much smaller than expected for a dog of his age and breed. In contrast, his weight was above the average for his age/breed group. At the end of the experiment, the dog was castrated and histological evaluations of the testes were performed as described below. Microscopic evaluation of the testes showed bilateral degeneration of seminiferous tubules with loss of germinal cells and/or maturation arrest of spermatogenesis so that no mature sperm were present within the seminiferous tubules or the epididymis. Such lesions are consistent with those seen in anti-GnRH preparations (for example: Jinshu et al. Vaccine 2005 23(40):4834-43.

Histopathology Examination of the Immunized Dog:

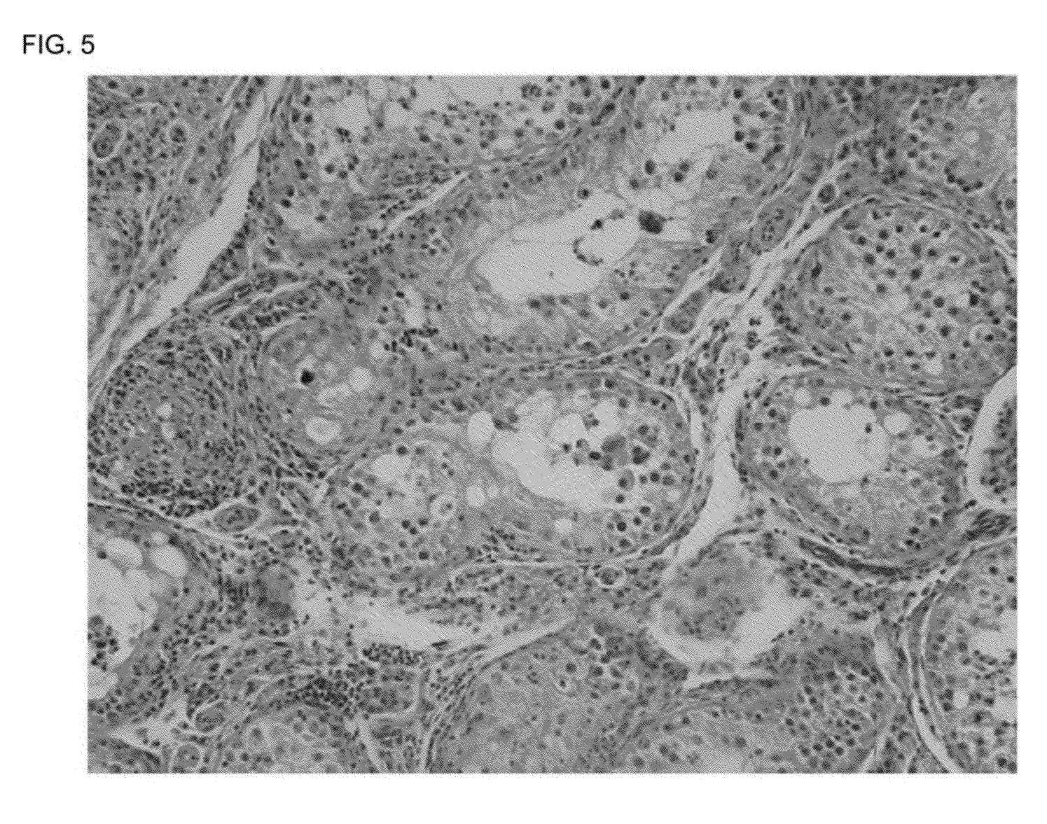

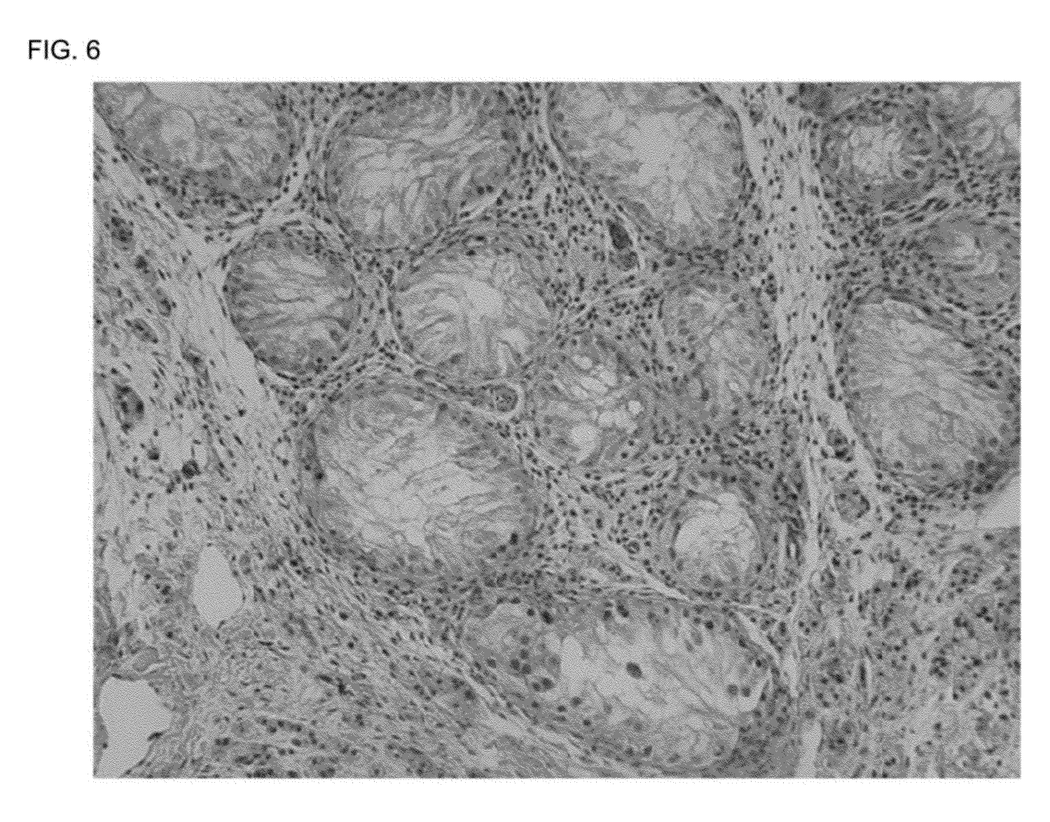

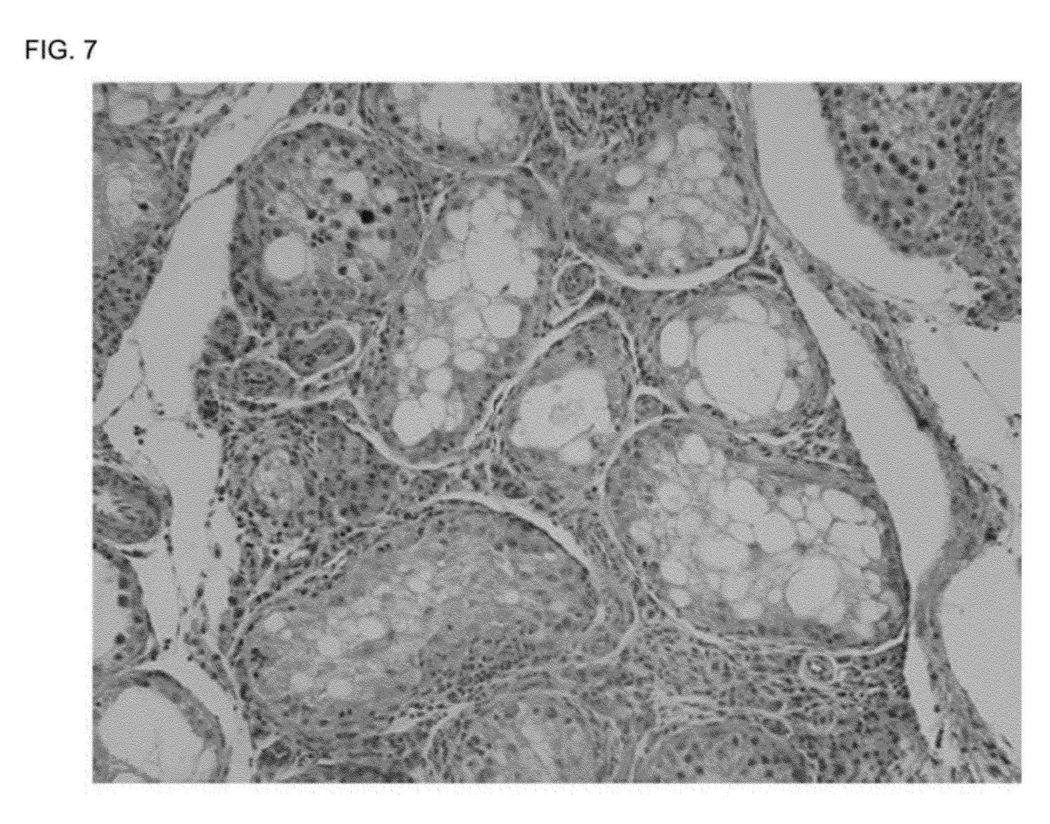

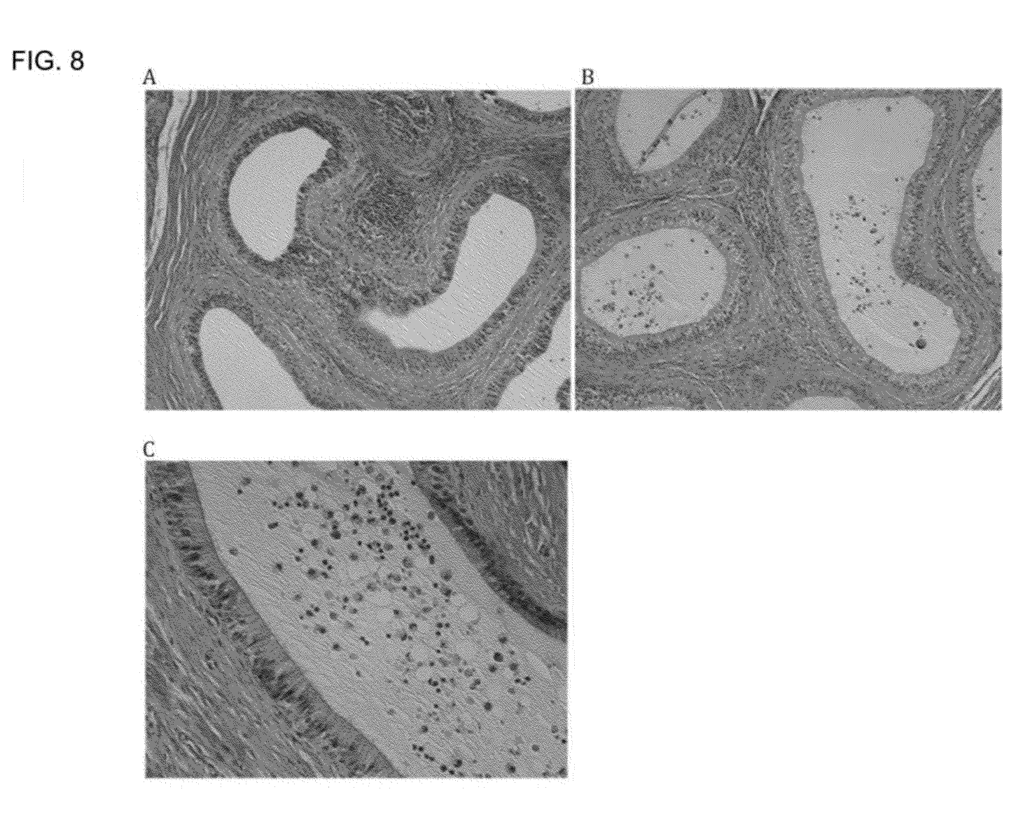

A histopathology examination was performed on the left testicle and right testicle of the immunized dog (male beagle age 2.5 years). The testicle tissue was fixed in 4% paraformaldehyde. (FIGS. 5-8).

On gross examination of slides, each block contained longitudinal sections of testicles with associated epididymal structures. Both testicles appear smaller than normal when compared to size of epididymis. However, this may be in part due to the gross sectioning as the mediastinum testis (central connective tissue cord extending through testis on its long axis is not prominent, suggesting sections are taken slightly off midline of the long axis in each of the blocks.) Four blocks of tissue were analyzed.

Microscopic findings: All blocks showed similar changes including seminiferous tubule degeneration and multifocal perivascular and peritubular inflammation. Testicular tissue was almost completely surrounded by a connective tissue capsule (tunica albuginea and tunica vaginalis). There was a small area where the capsule and some of the adjacent testis had been cut to allow fixative to reach interior tissues. The connective tissue was normal in appearance and contained numerous variably sized vessels. Within the testes, seminiferous tubules were separated by bands of interstitial tissue as the tubules course in a serpentine manner toward the mediastinum. The interstitial tissue was not extensive and there was some shrinkage due to paraformaldehyde fixation. The interstium contained numerous small vessels, lymphatics, nerves, and several types of cells including fibrocytes. Some of the cells were the interstitial cells of Leydig characterized by large round nuclei and clear to moderately granular cytoplasm. There were also small multifocal accumulations of mononuclear cells (lymphocytes and plasma cells) with occasional neutrophils within the connective tissue. These inflammatory foci were located primarily around small vessels, but occasionally surround degenerating or small seminiferous tubules.

Seminiferous tubules were surrounded by peritubular connective tissue with a basement membrane abutting the tubule epithelial cells of the tubules. The tubules varied in diameter and in some areas the basement membrane appeared to be undulating, suggestive of tubular degeneration. Some tubules showed thickened peritubular connective tissue in association with peritubular inflammation. Within the tubular epithelium itself, Sertoli cells were frequently vacuolated, sometimes to an extreme amount and there was no evidence of spermatogenesis past the spermatocyte stage, although spermatogonia and primary spermatocytes were present in variable amounts within tubules (consistent with maturational arrest of spermatogenesis). Nuclei within the epithelial cells of the tubules was either prominent, faded, condensed into apoptotic bodies, or not present at all, also suggesting degeneration has or is occurring. In some tubules, there were exfoliated cells, cellular debris and/or proteinaceous material. Other tubules appear empty; still other tubules appear collapsed. There were no spermatozoa present.

Epididymal tubules were also devoid of spermatozoa. Occasional cellular debris was present within epididymal tubules. Additionally, there were moderate numbers of inflammatory foci (granulomatous with occasional neutrophils) within the adjacent connective tissue, also in association with small vessels. A single focus contained neutrophils and abundant plasma cells and may have obliterated a tubule. These appeared to be more prominent in areas adjacent to smaller diameter tubules with columnar epithelium (probable head of epididymis). In some areas, peritubular connective tissue was increased in diameter.

Gross diagnosis: Testicular atrophy, bilateral.

Microscopic diagnosis: Testes, bilateral, seminiferous tubules, degeneration, widespread, moderate to severe, with lack of mature spermatocytes and spermatids (maturational arrest); Testes, bilateral, interstial inflammation, multifocal, subacute to chronic. Epididymis, bilateral, aspermia; Epididymis, bilateral, inflammation, subacute to chronic, multifocal, perivascular and peritubular.

Summary: Degenerative lesions within the testicles and lack of spermatozoa formation were consistent with testicular degeneration seen with the loss or reduction of GnRH or leutinizing hormone (LH). Total duration of spermatogenesis was presumed to be approximately 62 days in a dog and proliferating spermatozoa were presumed to divide at a fixed time interval of 13.6±0.7 days.

### REFERENCES

1. Brigati J R, Petrenko V A. Thermostability of landscape phage probes. Anal Bioanal Chem. 2005 July; 382(6):1346-50.
2. Eriksson F, Culp W D, MaSsey R, Egevad L, Garland O, Persson M A, Pisa P. Tumor specific phage particles promote tumor regression in a mouse melanoma model. Cancer Immunollmmunother. 2007 May; 56(5):677-87.
3. Eriksson F, Tsagozis P, Lundberg K, Parsa R, Mangsbo S M, Persson M A, Harris R A, Pisa P. Tumor-specific bacteriophages induce tumor destruction through activation of tumor-associated macrophages. J. Immunol. 2009 Mar. 1; 182(5):3105-11.
4. Fang J, Wang G, Yang Q, Song J, Wang Y, Wang I. The potential of phage display virions expressing malignant tumor specific antigen MAGE-A1 epitope in murine model. Vaccine. 2005 Sep. 23; 23(40):4860-6.
5. Frenkel O, Dewachter I, Van Leuven F, Solomon B. Reduction of beta-amyloid plaques in brain of transgenic mouse model of Alzheimer's disease by EFRH-phage immunization. Vaccine. 2003 Mar. 7; 21 (11-12): 1 060-5.
6. Houimel M, Dellagi K. Peptide mimotopes of rabies virus glycoprotein with immunogenic activity. Vaccine. 2009 Jul. 3; 27(34):4648-55.
7. Jung M J, Moon Y C, Cho I H, Yeh J Y, Kim S E, Chang W S, Park S Y, Song C S, Kim H Y, Park K K, McOrist S, Choi I S, Lee J B. Induction of castration by immunization of male dogs with recombinant gonadotropin-releasing hormone (GnRH) canine distemper virus (CDV) T helper cell epitope p35. J Vet Sci. 2005 March; 6(1):21-4.
8. Khan M A, Ogita K, Ferro V A, Kumasawa K, Tsutsui T, Kimura T. Immunisation with a plasmid DNA vaccine encoding gonadotrophin releasing hormone (GnRH-I) and T-helper epitopes in saline suppresses rodent fertility. Vaccine. 2008 March; 26(10): 1365-74
9. Killian G, Miller L, Rhyan J, Doten H. Immunocontraception of Florida feral swine with a single-dose GnRH vaccine. Am J Reprod Immunol. 2006 May; 55(5):378-84.
10. Kutzler M, Wood A. Non-surgical methods of contraception and sterilization. Theriogenology. 2006 August; 66(3):514-25.
11. Levy J K, Miller L A, Cynda Crawford P, Ritchey J W, Ross M K, Fagerstone K A. GnRH immunocontraception of male cats. Theriogenology. 2004 Sep. 15; 62(6):1116-30.
12. Manoutcharian K, Gevorkian G, Cano A, Almagro J C. Phage displayed biomolecules as preventive and therapeutic agents. Curr Pharm Biotechnol. 2001 September; 2(3):217-23.
13. Miller L A, Gionfriddo J P, Fagerstone K A, Rhyan J C, Killian G J. The single-shot GnRH immunocontraceptive vaccine (GonaCon) in white-tailed deer: comparison of several GnRH preparations. Am J Reprod Immunol. 2008 September; 60(3):214-23.
14. Minenkova A A, Ilyichev A A, Kishchenko G P, Petrenko V A. Design of specific immunogens using filamentous phage as the carrier. Gen. 1993:85-88.
15. Oonk H B, Turkstra J A, Schaaper W M, Erkens J H, Schuitemaker-de Weerd M H, van Nes A, Verheijden J H, Meloen R H. New GnRH-like peptide construct to optimize efficient immunocastration of male pigs by immunoneutralization of GnRH. Vaccine. 1998 July; 16(11-12):1 074-82. September; 9(9):797-801.
17. Wang G, Sun M, Fang J, Yang Q, Tong H, Wang L. Protective immune responses against systemic candidiasis mediated by phage-displayed specific epitope of Candida albicans heat shock protein 90 in C57BL16J mice. Vaccine. 2006 Aug. 28; 24(35-36):6065-73.
18. Yang Q, Su Q P, Wang G Y, Wen O Z, Zhang Y H, Bao H Z, Wang L. Production of hybrid phage displaying secreted aspartyl proteinase epitope of Candida albicans and its application for the diagnosis of disseminated candidiasis. Mycoses. 2007 May; 50(3):165-71.
19. Yip Y L, Smith G. Ward R L. Comparison of phage pili, pVIII and GST as carrier proteins for peptide immunisation in Balb/c mice. Immunol Lett, 2001 Dec. 3; 79(3): 197-202.
20. Zeng X Y, Turlstra J A, van de Wiel O F, Otto D Z, Liu X Y, Meloen R H, Schaaper W M, Chen F Q, Oonk H B, Zhang X. Active immunization against gonadotrophin-releasing hormone in Chinese male pigs. Reprod Domest Anim. 2001 April; 36(2): 1 01-5.
21. Zuercher A W, Miescher S M, Vogel M, Rudolf M P, Stadler M B, Stadler B M. Oral anti-IgE immunization with epitopedisplaying phage. Eur J Immunol. 2000 January; 30(1):128-35.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for obtaining an antigen for generating an immune response against gonadotropin releasing hormone (GnRH), the method comprising:
(a) contacting a phage library with anti-GnRH antibodies; and
(b) selecting phage from the library that hind specifically to the anti-GnRH antibodies.

2. The method of claim 1, wherein the phage library is first contacted with control antibodies and phage that bind to the control antibodies are removed from the phage library.

3. An isolated recombinant filamentous phage comprising a heterologous peptide, wherein the phage binds to an antibody against GnRH.

4. The phage of claim 3, wherein the heterologous peptide is at least about eight amino acids in length.

5. The phage of claim 3, wherein the heterologous peptide comprises amino acid sequence EH.

6. The phage of claim 3, wherein the heterologous peptide comprises amino acid sequence EHWS (SEQ ID NO:2).

7. The phage of claim 3, wherein the heterologous peptide comprises amino acid sequence EWS.

8. The phage of claim 3, wherein the heterologous peptide comprises amino acid sequence RPG.

9. The phage of claim 3, wherein the heterologous peptide comprises amino acid sequence EHWS (SEQ ID NO:2) and amino acid sequence RPG.

10. The phage of claim 3, wherein the heterologous peptide comprises an amino acid sequence having at least about 50% sequence identity to EHWSYGLRPG (SEQ ID NO:1).

11. The phage of claim 3, wherein the heterologous peptide comprises an amino acid sequence having at least about 50% sequence identity to EHPAGMTGD (SEQ ID NO:3).

12. The phage of claim 3, wherein the heterologous peptide comprises an amino acid sequence having at least about 50% sequence identity to EHWSYGLDPA (SEQ ID NO:6).

13. The phage of claim 3, wherein the heterologous peptide is inserted within a pVIII protein of the bacteriophage.

14. The phage of claim 13, wherein the filamentous bacteriophage has no more than a single gene 8.

15. An immunogenic composition comprising:
(a) the phage of claim 3; and
(b) a suitable excipient, carrier, or diluent.

16. The composition of claim 15, further comprising an adjuvant.

17. A method for producing antibodies that bind to GnRH, the method comprising administering the immunogenic composition of claim 15 to an animal.

18. A method for immunizing an animal against conception, the method comprising administering the immunogenic composition of claim 15 to the animal.

19. An isolated polynucleotide comprising genomic nucleic acid of the recombinant bacteriophage of claim 3.

20. An isolated peptide comprising an amino acid sequence selected from a group consisting of SEQ ID NOS:1-29.
